(19) United States
(12) Patent Application Publication
(10) Pub. No.: US 2020/0108182 A1
(43) Pub. Date: Apr. 9, 2020

(54) SELF-EXPANDABLE STENT AND METHOD OF PRODUCING THE SAME

(71) Applicant: TERUMO KABUSHIKI KAISHA, Tokyo (JP)

(72) Inventors: Shuhei MATSUSHITA, Hadano-shi (JP); Kazuyoshi TANI, Kawasaki-shi (JP)

(73) Assignee: TERUMO KABUSHIKI KAISHA, Tokyo (JP)

(21) Appl. No.: 16/703,086

(22) Filed: Dec. 4, 2019

Related U.S. Application Data
(63) Continuation of application No. PCT/JP2018/025582, filed on Jul. 5, 2018.

(30) Foreign Application Priority Data
Jul. 14, 2017 (JP) 2017-138397

(51) Int. Cl. A61L 31/06; A61F 2/90; A61F 2/844

(52) U.S. Cl. CPC A61L 31/06; A61F 2/844; A61F 2/90

(57) ABSTRACT

A self-expandable stent which has sufficient radial force, good flexural properties, and recovers the shape from a diameter in a contracted state to a diameter before contraction in a short period of time at approximately body temperature (37° C.). The self-expandable stent includes a crosslinked polymer that includes constitutional unit (A), which is a rigid biodegradable polymer derived from a first monomer; constitutional unit (B), which is a rubber-like biodegradable polymer derived from a second monomer; and constitutional unit (C) derived from a crosslinking agent. The constitutional unit (C) is present in an amount that is equal to or greater than 10% by weight and less than 60% by weight based on the total amount of constitutional units (A) and (B).

### CROSS-REFERENCES TO RELATED APPLICATIONS

This application is a continuation of International Application No. PCT/JP2018/025582 filed on Jul. 5, 2018, which claims priority to Japanese Application No. 2017-138397 filed on Jul. 14, 2017, the entire content of both of which is incorporated herein by reference.

### TECHNICAL FIELD

The present invention generally relates to a self-expandable stent and a method of producing the same.

### BACKGROUND DISCUSSION

A stent is a medical device that is used for expanding a stenosed or obstructed site to secure a lumen in order to treat various diseases caused by stenosis or obstruction of a lumen, such as a blood vessel. In recent years, acute myocardial infarction (AMI) has been also treated using a stent. In a treatment of AMI which is a thrombotic lesion using a stent, incomplete stent apposition (ISA) of the stent to a vascular wall is likely to occur due to thrombolysis after indwelling the stent.

Note that stents include balloon-expandable stents that are expanded with a balloon to which the stent is mounted and self-expandable stents that expand by itself by removal of a member that suppresses the expansion from the outside.

A self-expandable stent is stored in a delivery system, such as a sheath, in a contracted state, and on reaching the site to be indwelled, the stent is released from restriction to thereby self-expand. Thus an expansion operation, which is performed when a balloon-expandable stent is indwelled, is not required. As such a self-expandable stent, a self-expandable stent formed of a super-elastic alloy, such as a nickel-titanium alloy, is commercially available in Europe. Through a treatment with such a self-expandable stent, short-term incomplete stent apposition in AMI treatments has been dramatically improved.

As a material for forming a stent, nickel-titanium or other super-elastic alloys, which have a strong radial force (expansion retention force in the radial direction), are effective in that a vascular wall is maintained in a given diameter during the treatment. However, since a strong radial force is applied to the vascular wall over a long time after the period of treatment, major adverse cardiac events (MACE), in particular, target lesion revascularization (TLR), in the medium to long-term clinical outcomes are sometimes inferior to the case of a balloon-expandable stent.

In view of the above situation, a stent formed of a biodegradable material has been developed. Since a biodegradable material is gradually degraded in a living body, it is supposed that the radial force of the stent decreases over time to improve the medium to long-term clinical outcomes (particularly TLR).

As such a biodegradable stent, Japanese Patent Application Publication No. 2015-527920 (International Patent Application Publication No. 2014/018123) discloses a stent formed of a shape-memory random copolymer composed of poly(L-lactide) (PLLA) and a rubber-like polymer.

In addition, U.S. Patent Application Publication No. 2010/0262223 discloses a method of producing a stent, the method including crosslinking a biodegradable polymer by a crosslinking agent to form a base material.

### SUMMARY

However, the stent disclosed in Japanese Patent Application Publication No. 2015-527920 (International Patent Application Publication No. 2014/018123) requires expansion with a balloon catheter in order to change the crimped state (contracted state) to the expanded state. Furthermore, [0119] in Japanese Patent Application Publication No. 2015-527920 (p38, I2-8 in International Patent Application Publication No. 2014/018123) has a statement that a stent fabricated using a resin composed of 90:10 (by mole) of polylactic acid and polycaprolactone inwardly recoils for 60 minutes after expansion and then outwardly recoils (recovers the shape) over several days. When the shape recovery is so slow as in the above case, incomplete stent apposition of the stent is likely to occur. The incomplete stent apposition of a stent may lead to onset of stent thrombosis and in some cases may lead to movement of the stent by blood flow. Accordingly, a rapid shape recovery for returning from a diameter in a contracted state to a diameter before contraction in a short period of time is required for a self-expandable stent.

In contrast, as disclosed in [0049] in Japanese Patent Application Publication No. 2015-527920 (p18, I14-21 in International Patent Application Publication No. 2014/018123), an increased amount of a rubber-like polymer blended in a copolymer of PLLA and the rubber-like polymer is considered to lead to improvement of elastic properties of the polymer and reduction of the inward recoil. However, when the rubber-like polymer is merely increased in the resin, the stent does not have enough strength in the radial direction to support a stenosed artery.

In addition, U.S. Patent Application Publication No. 2010/0262223 discloses a tendency of a stent to undergo self-expansion, but does not discuss the speed of the shape recovery for outwardly expanding from a contracted state around body temperature (37° C.). Furthermore, in U.S. Patent Application Publication No. 2010/0262223, a self-crosslinking-type polymer formed of L-lactide and α-allyl-σ-valerolactone or a self-crosslinking-type polymer formed of L-lactide-α,α-diallyl-σ-valerolactone is merely produced and no specific study is made about the specific characteristics thereof.

Furthermore, when a stent is contracted in diameter from an expanded state to a crimped state, a local stress of approximately 10% is applied to each of the tensile direction and the compression direction in the vicinity of the folding points (apexes of the zigzag) of a stent strut, generating a strain. Thus, resistance to the generated strain is also required.

Accordingly, a self-expandable stent that has sufficient radial force, has sufficient strain resistance, and exhibits rapid shape recovery for outwardly expanding from a contracted state when released from restriction around body temperature (37° C.) is disclosed.

A self-expandable stent includes a crosslinked polymer. The crosslinked polymer includes a constitutional unit (A) that is a rigid biodegradable polymer derived from a first monomer, a constitutional unit (B) that is a rubber-like biodegradable polymer derived from a second monomer, and a constitutional unit (C) derived from a crosslinking agent. The constitutional unit (C) is present in an amount that is equal to or greater than 10% by weight and less than 60% by weight based on the total amount of the constitutional units (A) and (B).

According to another aspect, a method of producing a self-expandable stent is provided. The method includes polymerizing a copolymer that includes constitutional units (A) and (B) with a crosslinking agent. Constitutional unit (A) is a rigid biodegradable polymer derived from a first monomer. Constitutional unit (B) is a rubber-like biodegradable polymer derived from a second monomer. The crosslinking agent is present in an amount that is equal to or greater than 10% by weight and less than 60% by weight based on the amount of the copolymer used to produce a crosslinked polymer. The method further includes fabricating the stent using the crosslinked polymer.

According to another aspect, a self-expandable stent is provided that includes constitutional units (A), (B) and (C). Constitutional unit (A) is a rigid biodegradable polymer derived from a first monomer selected from the group consisting of L-lactic acid, D-lactic acid and glycolic acid; constitutional unit (B). Constitutional unit (B) is a rubber-like biodegradable polymer derived from a second monomer selected from the group consisting of ε-caprolactone, σ-butyrolactone, σ-valerolactone, 4-hydroxybytyrate, 3-hydroxybytyrate, 3-hydroxyvalerate, trimethylene carbonate, ethylene succinate, butylene succinate, and p-dioxanone. Constitutional unit (C) is derived from a crosslinking agent.

### BRIEF DESCRIPTION OF DRAWING FIGURES

FIGS. 1(A) and 1(B) illustrate a stent according to an embodiment, wherein FIG. 1(A) is a development of the stent and FIG. 1(B) is a partial enlargement of FIG. 1(A). In FIGS. 1(A) and 1(B), 3 denotes a first wave-shaped strut, 4 denotes a second wave-shaped strut, 5 denotes a connecting strut, 6 denotes a joint, 7 denotes a radiopaque marker, 8 denotes a bulging portion, 9 and 51 each denote a bent portion, 10 denotes a stent, 30 denotes a stent base, 35a and 45a each denote a slightly-bent portion, 38 denotes a top point of the first wave-shaped strut 3, 39 denotes a bottom point of the first wave-shaped strut 3, 48 denotes a bottom point of the second wave-shaped strut 4, and 49 denotes a top point of the second wave-shaped strut 4.

FIG. 2 is a graph showing the stroke displacement (stroke, length of displacement) over time in a tensile test for explaining a recovery rate.

FIG. 3 is a stress-stroke displacement chart in a tensile test for explaining a recovery rate.

FIG. 4 is a graph showing measurement results of radial force for stents of Example 25 and Comparative Example 7.

### DETAILED DESCRIPTION

Set forth below with reference to the accompanying drawings is a detailed description of embodiments of a self-expandable stent and a method of fabricating such stent representing examples of the inventive stent and manufacturing method disclosed here. Embodiments are described below, though the present invention is not limited only to the following embodiments. As used herein, “X to Y” for expressing a range means “X or more and Y or less” and unless otherwise specified, operations and measurements of physical properties are carried under conditions of room temperature (20 to 25° C.) and a relative humidity of 40 to 50% RH.

A first embodiment is a self-expandable stent that contains a crosslinked polymer containing a constitutional unit (A) derived from a monomer to constitute a rigid biodegradable polymer, a constitutional unit (B) derived from a monomer to constitute a rubber-like biodegradable polymer, and a constitutional unit (C) derived from a crosslinking agent, the constitutional unit (C) being contained in an amount of 10% by weight or more and less than 60% by weight based on the total amount of the constitutional unit (A) and the constitutional unit (B).

This embodiment provides a self-expandable stent that rapidly recovers its shape after the stent is released from restriction in a contracted state, has sufficient resistance to local stress on a stent strut that is generated when the stent is contracted in diameter from an expanded state to a crimped or contracted state, and has increased radial force.

Hereinafter, the constitutional unit (A) derived from a monomer to constitute a rigid biodegradable polymer is referred to as a constitutional unit (A), the constitutional unit (B) derived from a monomer to constitute a rubber-like biodegradable polymer is referred to as a constitutional unit (B), and the constitutional unit (C) derived from a crosslinking agent is referred to as a constitutional unit (C).

The crosslinked polymer has a structure in which a copolymer containing the constitutional unit (A) and the constitutional unit (B) is crosslinked by a constitutional unit (C) derived from a crosslinking agent.

The constitutional unit (A), which is rigid, has rigidity around body temperature (37° C.). The constitutional unit (B), which is like rubber, has elasticity around body temperature (37° C.). Thus, the crosslinked polymer, which is a copolymer containing the constitutional unit (A) and the constitutional unit (B), has both properties of rigidity and elasticity (a property to return from a decreased diameter at insertion to a diameter before contraction) around body temperature. However, it is difficult to achieve both the properties at high levels. In the embodiment, since a copolymer is crosslinked by a crosslinking agent, the high rigidity and elasticity can both be achieved. Furthermore, since the crosslinking agent is contained in an amount of 10% by weight or more and less than 60% by weight based on the total amount of the constitutional unit (A) and the constitutional unit (B), the stent has sufficient resistance to strain due to stress locally applied on a stent strut when the stent is contracted in diameter from an expanded state to a crimped state.

Thus, with such a structure, the stent can self-expand instantly (for example, within 10 seconds) from a diameter (outer diameter) at insertion (a decreased diameter in a state where the stent is incorporated into a delivery system, for example, 1.5 mm) to an indwell diameter (an outer diameter immediately after indwell into a body, for example, 3.0 mm), and can further self-expand in a short time (for example, within 20 minutes) from the indwell diameter to the initial diameter (natural outer diameter in a state without restriction before incorporation into the delivery system, for example, 4.0 mm). In addition, since a sufficient strength in the radial direction can be exhibited, the expansion in the radial direction of a wall of a lumen, such as a blood vessel, can be maintained while the strength in the radial direction of the stent decreases in the process of healing due to the biodegradability. It is therefore supposed that according to the self-expandable stent of the first embodiment, the incomplete stent apposition of a stent is reduced and, moreover, the radial force is reduced as a resin degrades over time, to improve the medium to long-term clinical outcomes (in particular, TLR).

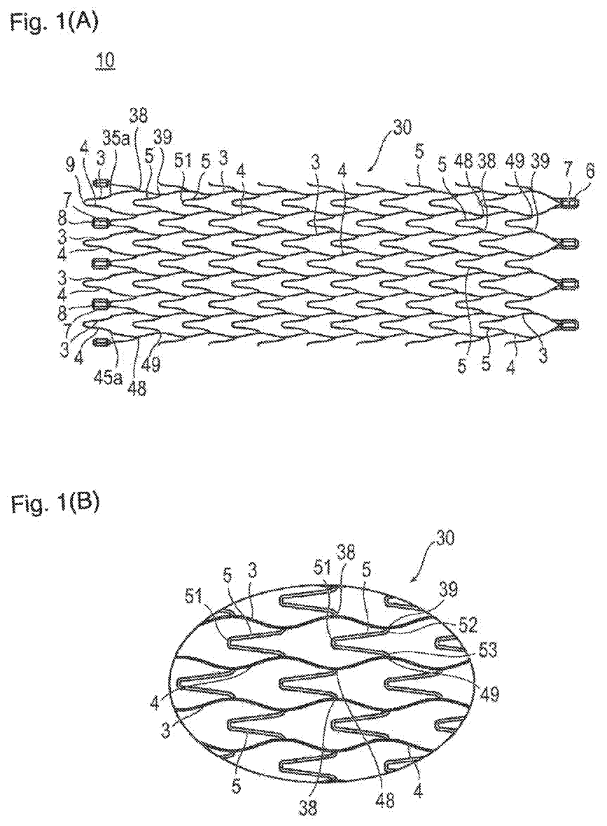

The stent of the embodiment will be described below with reference to the drawings. The ratios of sizes in the drawings are exaggerated for the sake of explanation and are sometimes different from the actual ratios. In the detailed description below, the longitudinal direction (right-left direction in FIG. 1(A)) of a stent is referred to as an axial direction.

First, components of the stent will be described. As an example of stent, a stent of FIGS. 7 and 8 of International Patent Application Publication No. 2011/034009 (U.S. Patent Application Publication No. 2012/0158119) is mentioned. The structure of a stent 10 explained by illustration in the drawings is only an example and the stent is not limited to the shape and structure (for example, arrangement and design of a strut) explained here.

As shown in FIGS. 1(A) and (B), the stent 10 according to one embodiment disclosed by way of example here includes a stent base (stent body) 30 and is formed in a contour of a substantially cylindrical shape having a prescribed length in the axial direction as a whole. The stent 10 is indwelled in a lumen (for example, blood vessel, bile duct, trachea, esophagus, other gastrointestinal tracts, and urethra) in a living body and is used for expanding the lumen to treat a stenosed or obstructed site. The stent 10 is a self-expandable stent that self-expands so that a stent base 30 assumes a previously-memorized shape with a prescribed expanded diameter after the start of the indwell. In addition, the stent 10 is a biodegradable stent which is degraded and absorbed in a living body. A strut which forms the stent base 30 of the stent 10 is formed of a biodegradable crosslinked polymer. The crosslinked polymer is degraded by, for example, hydrolysis, in a living body.

The stent base 30 includes multiple wave-shaped struts 3 and 4 that extend in the axial direction from one end to the other end of the stent base 30 and that are arranged in the peripheral or circumferential direction of the stent and multiple connecting struts 5 that connect circumferentially adjacent wave-shaped struts 3 and 4. The circumferentially adjacent wave-shaped struts 3 and 4 have multiple closer portions and multiple distant portions. That is, by virtue of the wave-shaped nature of the axially extending struts 3 and 4, some circumferentially adjacent portions of the wave-shaped struts 3 and 4 are positioned circumferentially closer to each other while other circumferentially adjacent portions of the wave-shaped struts 3 and 4 are positioned circumferentially farther away from each other as shown in FIGS. 1(A) and 1(B). The connecting struts 5 connect the circumferentially closer portions of the circumferentially adjacent wave-shaped struts 3 and 4. The connecting struts 5 each include at a central portion, a bent portion 51 extending in the axial direction of the stent. The bent portion 51 of each connecting strut 5 is a free end extends toward the distal end of the stent 10. The first wave-shaped struts 3 and the second wave-shaped struts 4 may have a shape of sine wave.

In the stent base 30, the first wave-shaped struts 3 and the second wave-shaped struts 4 have substantially the same wavelengths and substantially the same amplitudes, and the second wave-shaped struts 4 are shifted by about one-half wavelength in the axial direction of the stent with respect to the first wave-shaped struts 3.

Thus, as shown in FIG. 1(B), in the circumferentially adjacent first wave-shaped struts strut 3 and second wave-shaped struts 4, the top point 38 or the bottom point 39 of the first wave-shaped struts 3 and the bottom point 48 or the top point 49 of the second wave-shaped struts 4 substantially face each other to form the circumferentially closer portion and the wave-shaped distant portion.

In the stent base 30, the two ends 52 and 53 of each connecting strut 5 that are connected to the wave-shaped struts 3 and 4 are slightly-bent (curved) portions which curve outside the connecting strut 5 as shown in FIG. 1(B). The slightly-bent portion at one end 52 of each connecting strut 5 is connected to the top point 38 or the bottom point 39 of the wave-shaped strut 3 and the slightly-bent portion at the other end 53 of each connecting strut 5 is connected to the bottom point 48 or the top point 49 of the wave-shaped strut 4.

In the distal portion of the stent base 30, a bent portion 9 formed by connecting the distal portions of the first wave-shaped strut 3 and the second wave-shaped strut 4 and a bulging portion 8 provided in the bent portion 51 of the connecting strut 5 are alternately provided in the peripheral or circumferential direction. A radiopaque marker 7 as described later is attached or applied to the bulging portion 8. The bent portion 9 is positioned on the stent distal side with respect to the bulging portion 8. In this manner, a contrast marker on the distal side is positioned slightly on the inner side with respect to the stent end. Since the strut extends to an area outside the marker, the strut can securely cover a lesion area.

In the stent base 30, slightly-bent portions 35a and 45a which bend inside the bent portion 9 are provided at a given distance on the proximal side of the bent portion 9 formed by connecting the distal portions of the first wave-shaped strut 3 and the second wave-shaped strut 4 to thereby increase the expansion retention force of the bent portion 9 which is a long free end.

In the stent base 30, all the proximal portions of the first wave-shaped struts 3 and the second wave-shaped struts 4 are connected to joints 6 in the proximal portion of the stent. The stent base 30 has no free end that faces the proximal end of the stent except for the joints 6. Stated differently, all the bent portions face the distal end of the stent. Thus, when a sheath is moved toward the distal end relative to the stent, the stent is not hooked on the sheath because of no free end facing the sheath (stent storage member) so that it is possible to re-store the stent in a sheath (stent storage member).

In addition, the radiopaque marker 7 is attached to each joint 6. The joint 6 includes two frame portions that extend toward the end in parallel with a given distance therebetween and the radiopaque marker 7 covers substantially all or a part of the two frame portions. The radiopaque marker 7 has a thin rectangular parallelepiped shape, stores the two frame portions therein, and is fixed to the two frame portions by means of a recessed center thereof. Examples of materials that can be suitably used for forming the radiopaque marker include one (single element) or two or more (alloy) selected from the group of elements consisting of iridium, platinum, gold, rhenium, tungsten, palladium, rhodium, tantalum, silver, ruthenium, and hafnium.

The stents disclosed here include stents and stent grafts.

The thickness of the stent may be a conventionally common thickness. For example, the thickness of the stent is approximately from 50 to 500 μm, and in terms of the relationship between the supporting ability and the degradation time, the thickness is preferably approximately from 60 to 300 μm and more preferably approximately from 70 to 200 μm. Since the stent base according to the embodiment has superior physical properties (for example, expansion retention force), the thickness of the stent can be reduced.

The size of the stent is also appropriately adjusted according to the purpose and function thereof. For example, the outer diameter (the diameter) of the stent after expansion is preferably approximately from 1 to 40 mm, more preferably approximately from 1.5 to 10 mm, and most preferably approximately from 2 to 5 mm.

In addition, the length of the stent is not limited and can be appropriately selected depending on the disease to be treated. For example, the length is preferably approximately from 5 to 300 mm and more preferably approximately from 10 to 50 mm.

The stent base 30 is formed of a crosslinked polymer. The crosslinked polymer contains a constitutional unit (A) derived from a monomer to constitute a rigid biodegradable polymer, a constitutional unit (B) derived from a monomer to constitute a rubber-like biodegradable polymer, and a constitutional unit (C) derived from a crosslinking agent. The crosslinked polymer has a structure in which polymer chains are crosslinked by the constitutional unit (C), and specifically, the crosslinked polymer is preferably produced by polymerizing a copolymer containing the constitutional unit (A) and the constitutional unit (B) with a crosslinking agent of 10% by weight or more and less than 60% by weight based on the copolymer.

Regarding the rigid biodegradable polymer, “a polymer that is rigid” means a homopolymer produced by polymerizing a monomer and having a glass transition temperature (Tg) of 40° C. or higher. A polymer having a glass transition temperature of 40° C. or higher has rigidity at body temperature (approximately 37° C.). Thus, a copolymer having a monomer to constitute such a rigid biodegradable polymer can maintain a force in a radial direction even when indwelled in a lumen.

The glass transition temperature employed is a value measured by using Diamond DSC from Perkin Elmer according to JIS K7121:2012 (Testing Methods for Transition Temperatures of Plastics).

In addition, “biodegradable”, as used herein, means that in a biodegradability test described in Examples, the elongation at fracture after a hydrolysis test is 90% or less (lower limit 0%) of the elongation at fracture before the hydrolysis test.

Specific examples of monomers to constitute a rigid biodegradable polymer include L-lactic acid (Tg of poly(L-lactic acid) (PLLA): 60° C.), D-lactic acid (Tg of poly(D-lactic acid) (PDLA): 60° C.), and glycolic acid (Tg of poly(glycolic acid) (PGA): 45° C.). One of the monomers may be used alone, or two or more thereof may be used in combination. For example, L-lactic acid and glycolic acid may be used in combination, and L-lactic acid and D-lactic acid may be used in combination.

Among them, because of superior biodegradability and mechanical strength, the monomer to constitute a rigid biodegradable polymer is preferably lactic acid and/or glycolic acid, more preferably lactic acid, and most preferably L-lactic acid.

Note that in the case of lactic acid, since a direct polycondensation method cannot give a high molecular weight polymer, a lactide, which can be polymerized by ring-opening polymerization optionally in the presence of a catalyst, may be used. Lactides include L-lactide which is a cyclic dimer of L-lactic acid, D-lactide which is a cyclic dimer of D-lactic acid, meso-lactide which is a cyclic dimer of D-lactic acid and L-lactic acid, and DL-lactide which is a racemic mixture of D-lactide and L-lactide. Also for glycolic acid, a direct polycondensation method cannot produce a high molecular weight polymer, and thus ring-opening polymerization of glycolide can be used.

Regarding the rubber-like biodegradable polymer, “a polymer that is like rubber” refers to a homopolymer produced by polymerizing a monomer and having Tg of 30° C. or lower. A polymer having a glass transition temperature of 30° C. or lower has elasticity at body temperature (approximately 37° C.). Thus, a stent comprised of a copolymer containing a monomer to constitute such a rubber-like biodegradable polymer can return from a diameter-decreased state in a delivery system to a diameter before contraction after indwell.

Examples of monomers to constitute a rubber-like biodegradable polymer include lactone monomers, such as ε-caprolactone, σ-butyrolactone, and σ-valerolactone; hydroxyalkanoates, such as 4-hydroxybytyrate, 3-hydroxybytyrate, and 3-hydroxyvalerate; trimethylene carbonate, ethylene succinate, butylene succinate, and p-dioxanone. One of the monomers may be used alone, or two or more thereof may be used in combination.

Among them, because of degradability into a low-toxic monomer, the monomer to constitute a rubber-like biodegradable polymer is preferably ε-caprolactone (Tg of polycaprolactone −54° C.), p-dioxanone (Tg of polydioxanone −10° C.), or trimethylene carbonate (Tg of poly(trimethylene carbonate) −18° C.), and more preferably ε-caprolactone (Tg of polycaprolactone −54° C.) or trimethylene carbonate (Tg of poly(trimethylene carbonate) −18° C.), and most preferably ε-caprolactone.

In a suitable aspect of the embodiment, the monomer to constitute a rigid biodegradable polymer is lactic acid and the monomer to constitute a rubber-like biodegradable polymer is ε-caprolactone. Specifically, the copolymer containing the constitutional unit (A) and the constitutional unit (B) is preferably represented by —(C₃H₄O₂)ₙ—(C₆H₁₀O₂)ₘ—. Here, n and m represent molar fractions of the respective constitutional units in the polymer.

The constitutional ratio of the constitutional unit (A) and the constitutional unit (B) is not limited, but because of further rapid shape recovery and further increased radial force, the content of the constitutional unit (B) is preferably 10% by mole or more, and more preferably 10 to 60% by mole, based on the total amount of the constitutional unit (A) and the constitutional unit (B). The content of the constitutional unit (B) is further preferably 10 to 35% by mole, furthermore preferably 10 to 30% by mole, and particularly preferably 20 to 30% by mole, based on the total amount of the constitutional unit (A) and the constitutional unit (B). With a content of the constitutional unit (B) of 10% by mole or more, a stent that has improved elasticity and undergoes rapid shape recovery from a diameter at insertion can be obtained. With a content of the constitutional unit (B) of 60% by mole or less, the radial force can be secured. Note that the constitutional ratio of the constitutional units (A) and (B) is generally equal to the ratio of the respective monomers polymerized in production.

The copolymer containing the constitutional unit (A) and the constitutional unit (B) may be a random copolymer or may be a block copolymer. The block copolymer may be any of diblock (AB), triblock (ABA or BAB), multiblock (ABABA or BABAB), and the like.

The copolymer containing the constitutional unit (A) and the constitutional unit (B) may contain a constitutional unit derived from another monomer that is copolymerizable with the monomer to constitute a rubber-like biodegradable polymer and the monomer to constitute a rigid biodegradable polymer. In view of the effects of the embodiments, another such monomer is preferably contained in an amount of 5% by weight or less, more preferably 2% by weight or less, and most preferably is substantially not contained. “Substantially not contained” preferably means that the content is 0.01% by weight or less (lower limit 0% by weight).

Regarding a method of producing the copolymer containing the constitutional unit (A) and the constitutional unit (B), the copolymer can be produced by using conventionally known methods. For example, when the constitutional unit (A) is lactic acid and the constitutional unit (B) is ε-caprolactone, a polymerization reaction can be carried out using a lactide which is a cyclic dimer of lactic acid and ε-caprolactone as starting materials in the presence of a metal catalyst. Examples of metal catalysts include tin chloride, tin octylate, zinc chloride, zinc acetate, lead oxide, lead carbonate, titanium chloride, titanium alkoxide, germanium oxide, and zirconium oxide. The polymerization reaction may be carried out in the presence of an organic solvent. An initiator may be used in the polymerization reaction. Lactides include L-lactide which is a cyclic dimer of L-lactic acid, D-lactide which is a cyclic dimer of D-lactic acid, meso-lactide which is a cyclic dimer of D-lactic acid and L-lactic acid, and DL-lactide which is a racemic mixture of D-lactide and L-lactide. Any lactide can be used. In addition, two or more of the monomers may be combined to synthesize the copolymer.

From the viewpoints of improved mechanical strength and biodegradability, the copolymer preferably has an average molecular weight of 100,000 to 1,000,000, more preferably 150,000 to 800,000, and most preferably 150,000 to 600,000. Note that the average molecular weight in this description is a value measured by gel permeation chromatography (GPC) using polystyrenes as standard substances under the following measurement conditions.

[Conditions of Molecular Weight Measurement]

Apparatus: semi-micro GPC system LC-20AD (from Shimadzu Corporation)

Detector: Shodex (registered tradename) RI-104 (from Showa Denko K.K.)

Column: Shodex (registered tradename) GPC LF-404 (from Showa Denko K.K.)

Column temperature: 40° C.

Mobile phase solvent: CHCl₃

Flow rate: 0.15 mL/min

Injection: 20 μL

Sample preparation: 2 mL of the mobile phase solvent is added to 6 mg of a sample to be measured to dissolve the sample, followed by filtering through a 0.45-μm PTFE membrane filter.

As the copolymer containing the constitutional unit (A) and the constitutional unit (B), a commercial product may be used, and examples of commercial products include Resomer (registered tradename) LC703S, Resomer (registered tradename) DLCL9010, and Resomer (registered tradename) LT706S (all from EVONIK Industries AG); and BioDegmer (registered tradename) LCL (75:25), BioDegmer (registered tradename) LCL (60:40), BioDegmer (registered tradename) LCL (50:50), BioDegmer (registered tradename) LCL (90:10), and BioDegmer (registered tradename) LCL (65:35) (all from BMG Inc.).

A copolymer containing the constitutional unit (A) and the constitutional unit (B) is hereinafter also referred to as simply a copolymer.

The constitutional unit (C) is derived from a crosslinking agent.

The crosslinking agent in the constitutional unit (C) is preferably a monomer having two or more polymerizable unsaturated bonds. Examples of polymerizable unsaturated bonds include an acryloyl group (CH₂═CH—CO—), a methacryloyl group (CH₂═C(CH₃)—CO—), or a vinyl group (CH₂═CH—).

Specific examples of crosslinking agents include bifunctional (meth)acrylates, such as diethylene glycol diacrylate, 1,4-butanediol diacrylate, 1,3-butylene glycol diacrylate, dicyclopentanyl diacrylate, glycerol diacrylate, 1,6-hexanediol diacrylate, neopentyl glycol diacrylate, tetraethylene glycol diacrylate, ethylene glycol dimethacrylate, polyethylene glycol dimethacrylate, diethylene glycol dimethacrylate, triethylene glycol dimethacrylate, 1,4-butanediol dimethacrylate, 1,3-butylene glycol dimethacrylate, dicyclopentanyl dimethacrylate, glycerol dimethacrylate, 1,6-hexanediol dimethacrylate, neopentyl glycol dimethacrylate, tetraethylene glycol dimethacrylate, 1,9-nonanediol dimethacrylate, and 1,10-decanediol dimethacrylate; trifunctional (meth)acrylates, such as trimethylolpropane triacrylate, pentaerythritol triacrylate, tetramethylolmethane acrylate, trimethylolpropane trimethacrylate, and pentaerythritol trimethacrylate; tetra- or higher-functional (meth)acrylates, such as pentaerythritol tetraacrylate, ditrimethylolpropane tetraacrylate, dipentaerythritol hexaacrylate, dipentaerythritol monohydroxypentaacrylate, pentaerythritol tetramethacrylate, ditrimethylolpropane tetramethacrylate, dipentaerythritol hexamethacrylate, and dipentaerythritol monohydroxypentamethacrylate; N,N′-methylenebisacrylamide, N,N′-methylenebismethacrylamide, N,N′-ethylenebisacrylamide, N,N′-ethylenebismethacrylamide, N,N′-hexamethylenebisacrylamide, N,N′-hexamethylenebismethacrylamide, N,N′-benzylidenebisacrylamide, and N,N′-bis(acrylamidemethylene)urea; carboxylic acid allyl esters, such as trimellitic acid triallyl ester, pyromellitic acid triallyl ester, and diallyl oxalate; cyanuric acid or isocyanuric acid allyl esters, such as triallyl cyanurate and triallyl isocyanurate; maleimide compounds, such as N-phenylmaleimide and N,N′-m-phenylenebismaleimide; compounds having two or more triple bonds, such as dipropargyl phthalate and dipropargyl maleate; and divinylbenzene.

The crosslinking agent is more preferably a monomer having an acryloyl group (CH₂═CH—CO—) or a methacryloyl group (CH₂═C(CH₃)—CO—) because of further improved shape recovery. That is, the crosslinking agent is preferably a multifunctional (meth)acrylate. Furthermore, because of a superior initial radial force, the multifunctional (meth)acrylate crosslinking agent is more preferably a tetra- or higher-functional (meth)acrylate, and most preferably a tetra- to hexa-functional (meth)acrylate.

Among them, the crosslinking agent is preferably pentaerythritol tetraacrylate, ditrimethylol propane tetraacrylate, dipentaerythritol hexaacrylate, dipentaerythritol monohydroxypentaacrylate, pentaerythritol tetramethacrylate, ditrimethylolpropane tetramethacrylate, dipentaerythritol hexamethacrylate, and/or dipentaerythritol monohydroxypentamethacrylate, and more preferably pentaerythritol tetraacrylate and/or dipentaerythritol hexaacrylate.

In addition, the absolute value of the difference between the solubility parameter of the crosslinking agent and the weighted average of the solubility parameters of the constitutional unit (A) and the constitutional unit (B) is preferably 5 (J/cm³)^{1/2} or less. That is, in a suitable aspect of the first embodiment, the crosslinked polymer is produced by polymerizing the crosslinking agent with the copolymer containing the constitutional unit (A) derived from a monomer to constitute a rigid biodegradable polymer and the constitutional unit (B) derived from a monomer to constitute a rubber-like biodegradable polymer, and the absolute value of the difference between the solubility parameter of the crosslinking agent and the weighted average of the solubility parameters of the constitutional unit (A) and the constitutional unit (B) (hereinafter also referred to as solubility parameter difference) is 5 (J/cm³)^{1/2} or less. With a solubility parameter difference of 5 (J/cm³)^{1/2} or less, the effects of the embodiments (rapid shape recovery (high recovery rate) and high radial force (high Young's modulus)) are more easily obtained. This is because high compatibility of the crosslinking agent and the copolymer leads to a uniform crosslinking reaction. The solubility parameter difference is more preferably 2 (J/cm³)^{1/2} or less and most preferably 1.5 (J/cm³)^{1/2} or less. Note that the lower limit of the solubility parameter difference is zero.

“Solubility parameter (SP value)” refers to an SP value determined by a formula based on the Fedors method. Specifically, the SP value can be calculated by the following Formula (1) as described in Robert F Fedor, Poly Eng Sci 1974; 14(2):147-154.

SP=(ΔE_{V}/V)^{1/2} Formula (1)

In Formula (1), ΔEv represents the molar cohesive energy (the energy of vaporization at a given temperature) and V represents the molar volume. Note that in this application, “at a given temperature” means that the value was measured at 25° C.

The weighted average of the solubility parameters of the constitutional unit (A) and the constitutional unit (B) is determined as follows.

SP_{co}=SP_{A}×N_{A}+SP_{B}×N_{B} Formula (2)

SP_{co}: weighted average of solubility parameters of constitutional unit (A) and constitutional unit (B)
SP_{A}: SP value of constitutional unit (A)
SP_{B}: SP value of constitutional unit (B)
N_{A}: molar fraction of constitutional unit (A) in copolymer
N_{B}: molar fraction of constitutional unit (B) in copolymer

One of the crosslinking agents may be used alone, or two or more thereof may be used in combination.

The content of the constitutional unit (C) is 10% by weight or more and less than 60% by weight based on the total amount of the constitutional unit (A) and the constitutional unit (B). With a content of the constitutional unit (C) less than 10% by weight based on the total amount of the constitutional unit (A) and the constitutional unit (B), the Young's modulus significantly decreases and the expansion force in the radial direction cannot be maintained (Comparative Example 4 or Comparative Example 5 described later). In contrast, with a content of the constitutional unit (C) of 60% by weight or more based on the crosslinked polymer, the resin is brittle and the strain resistance is reduced (Comparative Example 3 described later). From the viewpoint of increased expansion force of a stent, the content of the constitutional unit (C) is preferably more than 10% by weight, more preferably 15% by weight or more, further preferably 20% by weight or more, and furthermore preferably 25% by weight or more, and particularly preferably 30% by weight or more based on the total amount of the constitutional unit (A) and the constitutional unit (B). From the viewpoint of the strain resistance, the content of the constitutional unit (C) is preferably 50% by weight or less, more preferably 45% by weight or less, and most preferably 40% by weight or less based on the total amount of the constitutional unit (A) and the constitutional unit (B). The content of the constitutional unit (C) is preferably 10 to 50% by weight, more preferably 10 to 45% by weight, further preferably 10 to 40% by weight, furthermore preferably 20 to 40% by weight, particularly preferably 25 to 40% by weight, and most preferably 30 to 40% by weight, based on the total amount of the constitutional unit (A) and the constitutional unit (B). When the crosslinking agent is a tetrafunctional (meth)acrylate, the content of the constitutional unit (C) is preferably 20 to 50% by weight and more preferably 30 to 50% by weight because of a high Young's modulus, and most preferably 30 to 40% by weight in view of the balance between the shape recovery and radial force, based on the total amount of the constitutional unit (A) and the constitutional unit (B). When the crosslinking agent is a hexafunctional (meth)acrylate, the content of the constitutional unit (C) is preferably 30 to 50% by weight based on the total amount of the constitutional unit (A) and the constitutional unit (B) because of a high Young's modulus.

Note that the content of the constitutional unit (C) corresponds to the amount of the crosslinking agent added in the production process. The content of the constitutional unit (C) can be determined by hydrolytically degrading the stent material to the monomer constitutional units and quantifying the monomer containing the constitutional unit (C) by HPLC.

The crosslinked polymer can be produced by polymerizing the copolymer with the crosslinking agent of 10% by weight or more and less than 60% by weight based on the copolymer. The method of producing the crosslinked polymer will be described later.

Crosslink refers to a chemical bond to bind one polymer chain to another polymer chain. As a non-limiting example, a C—H bond in a copolymer is cut by, for example, ultraviolet irradiation to generate a free radical site, and the free radical site reacts with an unsaturated bonding site in a crosslinking agent, thereby forming a structure in which the copolymer is crosslinked with the crosslinking agent.

The crosslinked polymer may contain, in addition to the constitutional units (A), (B), and (C), another biodegradable constitutional unit. Examples of compounds used for introducing another constitutional unit into a polymer include hydroxycarboxylic acids, dicarboxylic acids, polyhydric alcohols, and cyclic depsipeptide. The content of this added constitutional unit is preferably 0 to 10% by mole and more preferably 0 to 5% by mole based on all the constitutional units of the crosslinked polymer.

The Young's modulus of the crosslinked polymer is preferably 400 N/mm² or more, more preferably 500 N/mm² or more, and most preferably 550 N/mm² or more. With a Young's modulus within the above range, a high radial force is achieved and the mechanical strength is secured. The Young's modulus of the crosslinked polymer is further preferably 600 N/mm² or more, furthermore preferably 800 N/mm² or more, and most preferably 1000 N/mm² or more. A higher Young's modulus of the crosslinked polymer is more preferred. The upper value is not limited but is generally 3000 N/mm² or less. The Young's modulus of the crosslinked polymer can be controlled by the amount of the crosslinking agent added, the type of the crosslinking agent (combination of the crosslinking agent and the copolymer), and the like. The larger the amount of the crosslinking agent added to the copolymer, the larger the Young's modulus.

For the Young's modulus of the crosslinked polymer, a value measured by a method described later in Examples is employed.

The crosslinked polymer preferably has a recovery rate after 10 seconds of 70% or more. With a recovery rate after 10 seconds of 70% or more, the diameter can immediately return from a decreased diameter at insertion to a diameter before contraction, and expansion by a balloon catheter is not required and incomplete stent apposition is reduced. The recovery rate after 10 seconds is more preferably 72% or more and more preferably 75% or more. The upper limit of the recovery rate after 10 seconds is 100%, but generally is 95% or less. The recovery rate after 10 seconds can be controlled by the type and amount of the constitutional unit (B) and the type and amount of the constitutional unit (C). The higher the proportion of the constitutional unit (B), the higher the recovery rate after 10 seconds. Also, the higher the proportion of the constitutional unit (C), the higher the recovery rate after 10 seconds.

The recovery rate after 20 minutes of the crosslinked polymer is preferably 90% or more and more preferably 91.0% or more. With a recovery rate after 20 minutes of 90% or more, the stent can substantially return from a decreased diameter at insertion to a diameter before contraction and the incomplete stent apposition is reduced. The upper limit of the recovery rate after 20 minutes is 100%.

For the recovery rate after 10 seconds or the recovery rate after 20 minutes of the crosslinked polymer, a value measured by a method described later in Examples is employed.

In this description, a self-expansion stent refers to a stent in which the recovery rate after 20 minutes of the resin forming the stent base (for example, crosslinked polymer) is 70% or more.

In a suitable aspect of the embodiment, the Young's modulus of the crosslinked polymer is 500 N/mm² or more and the recovery rate after 10 seconds is 70% or more. In another suitable aspect of the embodiment, the Young's modulus of the crosslinked polymer is 600 N/mm² or more and the recovery rate after 10 seconds is 70% or more. In still another suitable aspect of the embodiment, the Young's modulus of the crosslinked polymer is 800 N/mm² or more and the recovery rate after 10 seconds is 70% or more. In still another suitable aspect of the embodiment, the Young's modulus of the crosslinked polymer is 800 N/mm² or more and the recovery rate after 10 seconds is 72% or more.

The glass transition temperature (Tg) of the crosslinked polymer is preferably 45° C. or lower and more preferably 40° C. or lower. With a glass transition temperature of the crosslinked polymer within the above temperature range, such a stent has elasticity at a temperature of approximately body temperature and therefore exhibits high recovery speed. Note that a lower glass transition temperature is more preferred, but the glass transition temperature is generally −50° C. or higher.

The crosslinked polymer has a gel fraction of 50% or more and more preferably 60% or more. With a gel fraction within the above range, the crosslinking sufficiently proceeds and a desired effect can be achieved. That is, the degree of crosslinking can be known through the gel fraction. The upper limit of the gel fraction is not limited, but is preferably 100% or less. For the gel fraction, a value measured by a method described later in Examples is employed.

Alternatively, the degree of crosslinking (for example, crosslinking density) in the crosslinked polymer can be measured by a method of tracing the degree of decrease in the peak of the heat of fusion by DSC.

Furthermore, the stent preferably has a Martens hardness in a loading-unloading test using a nanoindenter (hereinafter also referred to as simply Martens hardness) of 50 N/mm² or more. With a Martens hardness of 50 N/mm² or more, a high radial force is achieved and the mechanical strength is secured. The Martens hardness is preferably 100 N/mm² or more. A stent having a higher Martens hardness is more preferred, and the upper limit is generally, but not limited to, 200 N/mm² or less. The Martens hardness of the stent can be controlled by the amount of the crosslinking agent added, the type of the crosslinking agent (combination of the crosslinking agent and the copolymer), and the like. The larger the amount of the crosslinking agent added to the base polymer (copolymer), the larger the Martens hardness.

The crosslinked polymer preferably has lower crystallinity (higher amorphousness) in view of easy hydrolysis, namely, biodegradability. Thus, any operation for increasing the crystallinity, such as annealing, is not required in the production process.

A second embodiment is a method of producing a self-expandable stent including polymerizing a copolymer that contains a constitutional unit (A) derived from a monomer to constitute a rigid biodegradable polymer and a constitutional unit (B) derived from a monomer to constitute a rubber-like biodegradable polymer and a crosslinking agent of 10% by weight or more and less than 60% by weight based on the copolymer to produce a crosslinked polymer, and fabricating the stent using the crosslinked polymer.

The constitutional unit (A) derived from a monomer to constitute a rigid biodegradable polymer and the constitutional unit (B) derived from a monomer to constitute a rubber-like biodegradable polymer, and the copolymer containing the units are as previously described above.

In addition, specific examples of the crosslinking agents are as previously described above.

The polymerization of the copolymer and the crosslinking agent may be any mode, such as solution polymerization or bulk polymerization, but not limited thereto. The solvent used in the solution polymerization may be a solvent that can dissolve the copolymer and the crosslinking agent, and examples thereof include chloroform, 1,1,1,3,3,3-hexafluoro-2-propanol, and N—N-dimethylformamide.

The method of polymerization is preferably photopolymerization since unsaturated bonds can be easily activated. Examples of light (active radiations) used herein include ionizing radiations, such as electron beams, α-rays, β-rays, and γ-rays; and ultraviolet rays. Among them, the polymerization of a polymer and a crosslinking agent is preferably carried out under irradiation with ultraviolet rays because of simple production facility and easy production. The wavelength of the ultraviolet rays is preferably 200 to 400 nm. The quantity of the ultraviolet rays (integral light quantity) is appropriately set so that the polymerization is suitably achieved, and, for example, the quantity is 500 to 20,000 mJ/cm² and more preferably is 1,000 to 5,000 mJ/cm².

For enhancing polymerization efficiency, such polymerization under irradiation with ultraviolet rays is preferably carried out in the presence of a photoinitiator. The photoinitiator may be selected depending on the wavelength of the ultraviolet rays used, and may be benzyldimethylketal, any of alkylphenone compounds, such as α-hydroxyalkylphenones and α-aminoalkylphenones; acylphosphine oxide compounds, such as MAPO and BAPO; and oxime ester compounds. From the viewpoint of polymerization efficiency, an alkylphenone compound is preferred and an α-hydroxyalkylphenone is more preferred.

Specific examples of photoinitiators include α-hydroxyalkylphenones, such as 2-hydroxy-1-[4-(2-hydroxyethoxy)phenyl]-2-methyl-1-propanone, 2-hydroxy-2-methyl-1-phenyl-1-propanone, and 1-hydroxy-cyclohexyl-phenyl-ketone; α-aminoalkylphenones, such as 2-methyl-1-[4-methylthiophenyl]-2-morpholinopropan-1-one, 2-benzyl-2-dimethylamino-1-(4-morpholinophenyl)butanone-1, and 2-dimethylamino-2-(4-methylbenzyl)-1-(4-morpholin-4-ylphenyl)butan-1-one; and acylphosphineoxide compounds, such as diphenyl(2,4,6-trimethylbenzoyl)-phosphineoxide and phenylbis(2,4,6-trimethylbenzoyl).

As the photoinitiator, a commercial product may be used, and examples of commercial products include Irgacure (registered tradename) 2959, 184, 1173, 907, 369E, 379EG, TPO, and 819 (all from BASF).

One of the photoinitiators may be used alone, or two or more thereof may be used in combination.

The amount of the photoinitiator is preferably 1 to 20% by weight and more preferably 2 to 15% by weight based on the amount of the crosslinking agent.

The timing of irradiation with light is not limited and the irradiation may be carried out after molding a mixture containing the copolymer and the crosslinking agent into a tube shape by extrusion or injection molding. Note that processing into a desired stent shape may be achieved by laser cutting or the like. Furthermore, irradiation with light may be performed after molding a mixture containing the copolymer and the crosslinking agent into a tube shape by extrusion or injection molding and then processing the obtained tube into a desired stent shape by laser cutting or the like. Alternatively, irradiation with light may be performed after processing a mixture containing the copolymer and the crosslinking agent into a stent shape by injection molding or the like.

In the stent, any component other than the crosslinked polymer may be contained to the extent that the object and effects of the embodiments are not impaired. An example of the other component is a drug for suppressing stenosis or obstruction of the vascular system which possibly occurs when a stent is indwelled in a lesion area. Specific examples include anticancer agents, immunosuppressive agents, antibiotics, antithrombotic agents, HMG-CoA reductase inhibitors, ACE inhibitors, calcium antagonists, antilipemic agents, integrin inhibitors, antiallergic agents, antioxidants, GPIIb/IIIa antagonists, retinoids, lipid improvers, antiplatelet agents, and anti-inflammatory agents. These drugs have an advantage in that they are capable of controlling behavior of tissue cells in a lesion area to thereby treat the lesion area. The other component as previously described may constitute a stent base together with the crosslinked polymer or may be present as a coating layer over a stent base formed by the crosslinked polymer.

Preferred examples of anticancer agent include, but are not limited to, paclitaxel, docetaxel, vinblastine, vindesine, irinotecan, and pirarubicin.

Preferred examples of immunosuppressive agents include, but are not limited to, sirolimus derivatives, such as sirolimus, everolimus, pimecrolimus, and zotarolimus, biolimus (for example, Biolimus A9 (registered tradename)), tacrolimus, azathioprine, cyclosporine, cyclophosphamide, mycophenolate mofetil, and gusperimus.

Preferred examples of antibiotics include, but are not limited to, mitomycin, adriamycin, doxoruvicin, actinomycin, daunorubicin, idarubicin, pirarubicin, aclarubicin, epirubicin, and zinostatin stimalamer.

Preferred examples of antithrombotic agents include, but are not limited to, aspirin, ticlopidine, and argatroban.

Preferred examples of HMG-CoA reductase inhibitors include, but are not limited to, cerivastatin, cerivastatin sodium, atorvastatin, pitavastatin, fluvastatin, fluvastatin sodium, simvastatin, and lovastatin.

Preferred examples of ACE inhibitors include, but are not limited to, quinapril, trandolapril, temocapril, delapril, enalapril maleate, and captopril.

Preferred examples of calcium antagonists include, but are not limited to, nifedipine, nilvadipine, benidipine, and nisoldipine.

A preferred example of an antilipemic agent is, but is not limited to, probucol.

A preferred example of an integrin inhibitor is, but is not limited to, AJM300.

A preferred example of an antiallergic agent is, but is not limited to, tranilast.

Preferred examples of antioxidants include, but are not limited to, α-tocopherol, catechin, dibutylhydroxytoluene, and butylhydroxyanisole.

A preferred example of a GPIIb/IIIa antagonist is, but is not limited to, abciximab.

A preferred example of a retinoid is, but is not limited to, all trans retinoic acid.

A preferred example of a lipid improver is, but is not limited to, eicosapentaenoic acid.

Preferred examples of antiplatelet agents include, but are not limited to, ticlopidine, cilostazol, and clopidogrel.

Preferred examples of anti-inflammatory agents include, but are not limited to, steroids, such as dexamethasone and prednisolone.

When the stent contains any components other than the crosslinked polymer, the crosslinked polymer is contained in an amount of 80% by weight or more, preferably 90% by weight or more, most preferably 95% by weight or more (upper limit 100% by weight) in total based on the whole stent, and the balance is the other components.

The stent may be provided with a coating layer of any biodegradable material on a stent base in addition to the stent base to the extent that the object and effects of the embodiments are not impaired. Examples of biodegradable materials used for forming the coating layer include, but are not limited to, a polymer selected from the group consisting of polyesters, polyacid anhydrides, polycarbonates, polyphosphazenes, polyphosphoric acid esters, polypeptides, polysaccharides, proteins, and celluloses. Specific examples include at least one, or a blend of two or more selected from the group consisting of polylactic acids, polyglycolic acids, lactic acid-glycolic acid copolymers, polycaprolactone, lactic acid-caprolactone copolymers, polyhydroxybutyric acid, polymalic acid, poly-α-amino acids, collagen, laminin, heparan sulfate, fibronectin, vitronectin, chondroitin sulfate, and hyaluronic acid, and medically safe biodegradable materials are preferred in view of degradability in living bodies. The duration of strength can be prolonged by adjusting the molecular weight, degree of purification, degree of crystallization of the biodegradable material which coats a stent's outer surface (stent base outer surface) to suppress the hydrophilicity. For example, the time for hydrolysis can be prolonged, for example, by increasing the degree of purification of the biodegradable material to eliminate unreacted monomers and low molecular weight fractions or by increasing the degree of crystallization to suppress the amount of water permeating the inside of the stent backbone. The coating layer may also be a drug coating layer that contains the coating layer-forming biodegradable material and one or two or more of the aforementioned drugs at any ratio, for example, at 1:99 to 99:1 (w/w), and more preferably at 95:5 to 80:20 (w/w). The method of forming the coating layer is not limited and a common coating method can be adopted as is, or after appropriately being modified. Specifically, a method can be adopted in which a biodegradable material and, as needed, such a drug as previously discussed above and a suitable solvent are mixed to prepare a mixture and the mixture is applied on a stent base.

### EXAMPLES

The effects of the present invention are described with reference to Examples and Comparative Examples below. In Examples, expressions “parts” or “%” are sometimes used and, unless otherwise defined, represent “parts by weight” or “% by weight”. In addition, unless otherwise specified, various operations are carried out at a room temperature (25° C.).

### Example 1

1 g of L-Lactic acid/ε-caprolactone copolymer of L-lactic acid:ε-caprolactone=75:25 by mole (BMG Inc., BioDegmer (registered tradename) LCL (75:25), SP value: 22.6, molecular weight: 570,000), 0.1 g of pentaerythritol tetraacrylate (SP value: 21.5, PETA) (from Sigma-Aldrich) as a crosslinking agent, 0.01 g of 2-hydroxy-1-[4-(2-hydroxyethoxy)phenyl]-2-methyl-1-propanone (2-hydroxy-1-[4-(2-hydroxyethoxy)phenyl]-2-methyl-1-propanone) (Irgacure (registered tradename) 2959, from BASF), and 29.6 g of chloroform were mixed to prepare a polymer solution.

The polymer solution was poured into a ϕ100-mm PFA petri dish so as not to produce air bubbles and was dried with air over night at room temperature to obtain a cast film. The obtained film was irradiated with UV light of a wavelength of 365 nm from the front and back surface thereof in an integral light quantity of 3000 mJ/cm² using a UV irradiation device (VB-15201BY-A, from Ushio Inc.) to form a film (thickness: about 0.1 mm), which was then peeled from the petri dish to obtain a test film.

### Example 2

A test film was obtained in the same manner as in Example 1 except for changing the amount of the crosslinking agent from 0.1 g to 0.2 g.

### Example 3

A test film was obtained in the same manner as in Example 1 except for changing the amount of the crosslinking agent from 0.1 g to 0.3 g.

### Example 4

A test film was obtained in the same manner as in Example 1 except for changing the amount of the crosslinking agent from 0.1 g to 0.4 g.

### Example 5

A test film was obtained in the same manner as in Example 1 except for changing the amount of the crosslinking agent from 0.1 g to 0.5 g.

### Example 6

A test film was obtained in the same manner as in Example 2 except for changing the type of the crosslinking agent from pentaerythritol tetraacrylate to dipentaerythritol hexaacrylate (SP value: 22.5, DPEHA) (from Sigma-Aldrich).

### Example 7

A test film was obtained in the same manner as in Example 4 except for changing the type of the crosslinking agent from pentaerythritol tetraacrylate to dipentaerythritol hexaacrylate (SP value: 22.5, DPEHA) (from Sigma-Aldrich).

### Example 8

A test film was obtained in the same manner as in Example 2 except for changing the type of the crosslinking agent from pentaerythritol tetraacrylate to ethylene glycol dimethacrylate (SP value: 18.2, EGDM) (from Sigma-Aldrich).

### Example 9

A test film was obtained in the same manner as in Example 1 except for changing the type of the crosslinking agent from pentaerythritol tetraacrylate to ethylene glycol dimethacrylate (SP value: 18.2, EGDM) (from Sigma-Aldrich).

### Example 10

A test film was obtained in the same manner as in Example 9 except that no initiator was added and crosslinking was performed with an electron beam of 25 kGy.

### Example 11

A test film was obtained in the same manner as in Example 1 except for changing the L-lactic acid/ε-caprolactone copolymer of L-lactic acid:ε-caprolactone=75:25% by mole to an L-lactic acid/ε-caprolactone copolymer of L-lactic acid:ε-caprolactone=90:10% by mole (lactic acid-caprolactone copolymer, BMG Inc., BioDegmer (registered tradename) LCL (90:10), (SP value: 22.9, molecular weight: 530,000).

### Example 12

A test film was obtained in the same manner as in Example 11 except for changing the amount of the crosslinking agent from 0.1 g to 0.3 g.

### Example 13

A test film was obtained in the same manner as in Example 11 except for changing the amount of the crosslinking agent from 0.1 g to 0.4 g.

### Example 14

A test film was obtained in the same manner as in Example 2 except for changing the L-lactic acid/ε-caprolactone copolymer of L-lactic acid:ε-caprolactone=75:25% by mole to an L-lactic acid/ε-caprolactone copolymer of L-lactic acid:ε-caprolactone=65:35% by mole (lactic acid-caprolactone copolymer; BMG Inc., BioDegmer (registered tradename) LCL (65:35), (SP value: 22.4, molecular weight: 320,000).

### Example 15

A test film was obtained in the same manner as in Example 14 except for changing the amount of the crosslinking agent from 0.2 g to 0.3 g.

### Example 16

A test film was obtained in the same manner as in Example 14 except for changing the amount of the crosslinking agent from 0.2 g to 0.5 g.

### Example 17

A test film was obtained in the same manner as in Example 4 except for changing the L-lactic acid/ε-caprolactone copolymer of L-lactic acid:ε-caprolactone=75:25% by mole to an L-lactic acid/ε-caprolactone copolymer of L-lactic acid:ε-caprolactone=60:40% by mole (BMG Inc., BioDegmer (registered tradename) LCL (60:40), SP value: 22.3, molecular weight: 370,000).

### Example 18

A test film was obtained in the same manner as in Example 1 except for changing the type of the crosslinking agent from ethylene glycol dimethacrylate to triallyl isocyanate (SP value: 29.2, TAIC) (from Sigma-Aldrich).

### Example 19

A test film was obtained in the same manner as in Example 18 except for changing the amount of the crosslinking agent from 0.1 g to 0.3 g.

### Example 20

A test film was obtained in the same manner as in Example 18 except for changing the amount of the crosslinking agent from 0.1 g to 0.5 g.

### Example 21

A test film was obtained in the same manner as in Example 10 except for changing the type of the crosslinking agent from ethylene glycol dimethacrylate to triallyl isocyanate (SP value: 29.2, TAIC) (from Sigma-Aldrich).

### Example 22

A test film was obtained in the same manner as in Example 4 except for changing the L-lactic acid/ε-caprolactone copolymer of L-lactic acid:ε-caprolactone=75:25% by mole to a DL-lactic acid/ε-caprolactone copolymer of DL-lactic acid:ε-ca pro lactone=90:10% by mole (lactic acid-caprolactone copolymer, EVONIK Industries, trade name Resomer (registered tradename) DLCL9010, Mw: 180,000, lactic acid unit:caprolactone unit=90:10 (mol/mol)).

### Example 23

A test film was obtained in the same manner as in Example 3 except for changing the L-lactic acid/ε-caprolactone copolymer of L-lactic acid:ε-caprolactone=75:25% by mole to an L-lactic acid/trimethylene carbonate copolymer of L-lactic acid:trimethylene carbonate=70:30% by mole (EVONIK Industries, trade name Resomer (registered tradename) LT706S, Mw: 250,000, lactic acid unit:trimethylene carbonate unit=70:30 (mol/mol)).

### Comparative Example 1

1 g of poly-L-lactic acid (from BMG Inc., BioDegmer (registered tradename) PLLA, SP value: 23.1, weight average molecular weight: 510,000) and 29.6 g of chloroform were mixed to prepare a polymer solution. The obtained polymer solution was poured into a ϕ100-mm PFA petri dish so as not to produce air bubbles and was dried with air at room temperature, followed by drying under reduced pressured in a vacuum oven at 120° C. for 2 hours. The formed film (thickness: about 0.1 mm) was peeled from the PFA petri dish to obtain a test film.

### Comparative Example 2

A test film was obtained in the same manner as in Comparative Example 1 except for changing poly-L-lactic acid to an L-lactic acid (75% by mole)/ε-caprolactone (25% by mole) copolymer (BMG Inc., BioDegmer (registered tradename) LCL (75:25), SP value: 22.6, molecular weight: 570,000) used in Example 1 and changing the temperature and the time in the drying under reduced pressure to 80° C. and 2 hours, respectively.

### Comparative Example 3

A test film was obtained in the same manner as in Example 1 except for changing the amount of the crosslinking agent from 0.1 g to 0.6 g.

### Comparative Example 4

A test film was obtained in the same manner as in Example 1 except for changing the amount of the crosslinking agent from 0.1 g to 0.05 g.

### Comparative Example 5

A test film was obtained in the same manner as in Example 8 except for changing the amount of the crosslinking agent from 0.1 g to 0.05 g.

### Comparative Example 6

A test film was obtained in the same manner as in Comparative Example 2 except for changing the L-lactic acid/ε-caprolactone copolymer (BMG Inc., BioDegmer (registered tradename) LCL (75:25), SP value: 22.6, molecular weight: 570,000) to the L-lactic acid/trimethylene carbonate copolymer of L-lactic acid:trimethylene carbonate=70:30% by mole (EVONIK Industries, trade name Resomer (registered tradename) LT706S, Mw: 250,000, lactic acid unit:trimethylene carbonate unit=70:30 (mol/mol)) used in Example 23.

[Evaluation]

[Young's Modulus]

A 5B-type dumbbell test piece defined in ISO 527-2 was made with a punching die and then was subjected to a tensile test using a tensile tester equipped with a thermostatic chamber (Autograph AG-1kNIS, from Shimadzu Corporation) under an atmosphere of 37° C. at a distance between chucks of 20 mm and a testing speed of 1 mm/min, and the Young's modulus (MPa) was determined from an initial slope in an elastic deformation area of the stress-strain curve.

Recovery rate after 10 seconds, recovery rate after 20 minutes

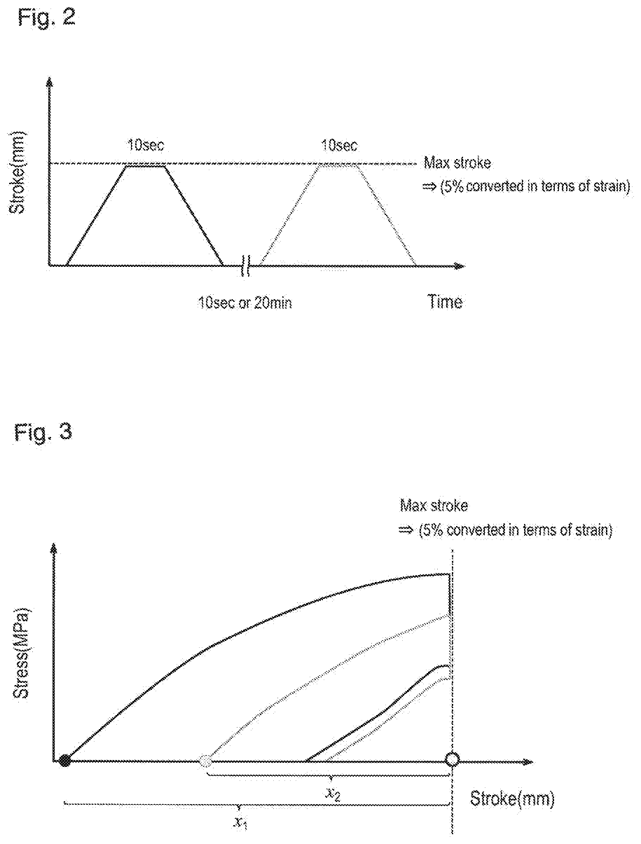

A 5B-type dumbbell test piece defined in ISO 527-2 was made with a punching die and then subjected to two cycles of a tensile test using a tensile tester equipped with a thermostatic chamber (Autograph AG-1kNIS, from Shimadzu Corporation) under an atmosphere of 37° C. at a distance between chucks of 20 mm, a testing speed of 10 mm/min, a maximum tensile distance of 0.6 mm (length of parallel part of dumbbell test piece 12 mm×5%), and a retention time of tensile strain of 10 seconds as shown in FIG. 2, and as shown in FIG. 3, the recovery rate was calculated as a ratio ((x₂/x₁)×100%) of the 2nd cycle elongation distance x₂ (distance from the strain-detected position to the maximum elongation position in the 2nd cycle) to the 1st cycle elongation distance x₁ (distance from the strain-detected position to the maximum elongation position in the 1st cycle). Note that the stand-by time between cycles was set to 10 seconds or 20 minutes.

The recovery rate measured in this test correlates to the degree of the shape recovery of the stent. When a stent is contracted in diameter by restriction with an external force, a strain in the tensile direction is generated on an outer curving side of an apex of a bent portion. On releasing the restriction in this state, since reduction in the strain occurs as found in this test, the stent diameter is allowed to return to the diameter before contraction. During this time, the higher the recovery rate, the more the strain is reduced and the closer the stent diameter becomes to the diameter before contraction. That is, the recovery rate correlates to the degree of shape recovery, and the higher the recovery rate, the higher the degree of shape recovery.

[Strain Resistance Property]

A 5B-type dumbbell test piece defined in ISO 527-2 was made with a punching die and then was subjected to a tensile test using a tensile tester equipped with a thermostatic chamber (Autograph AG-1kNIS, from Shimadzu Corporation) under an atmosphere of 37° C. at a diameter between chucks of 20 mm and a testing speed of 10 mm/min, and whether fracture of the sample occurred was determined at 1.8-mm elongation (length of parallel part of dumbbell test piece 12 mm×15%). A sample without fracture was rated as ∘ and a sample with fracture was as ×.

Note that in the diameter-decreased state, in the vicinity of a bent apex, the outer curving side was elongated, that is, has a strain in the tensile direction, and the inner curving side is compressed, that is, has a strain in the compression direction. Here, the strain resistance property when designed as a stent was evaluated by whether or not fracture occurred when a strain in the tensile direction was applied. Note that a stent designed as a self-expandable stent has a tensile strain on the outer curving side and a compression strain on the inner curving side of about 10% (at most 15% or less) in a diameter-decreased state, and thus, regarding the strain resistance property, such a stent that exhibits fracture at a 15%-strain possibly undergoes fracture in a diameter decreasing operation.

[Martens Hardness Test]

In accordance with ISO14577-1 “Instrumented Indentation Hardness”, a sheet surface was subjected to an indenter indentation test using a dynamic ultra micro hardness tester (DUH-W201S, from Shimadzu Corporation) under the following conditions: indenter: Berkovich indenter of a regular triangular pyramid shape with an intercristal angle of 115° (made of Diamond), testing force: 10 mN, loading rate: 0.473988 mN/sec, and retention time: 5 seconds, to obtain the indentation depth (μm) at this time, and the Martens hardness was determined based on the formula: [Martens hardness (N/mm²)]=1000×[load at the indentation depth (mN)]/26.43×[indentation depth (μm)]².

[Gel Fraction]

About 25 mg of each film was precisely weighed and immersed in 25 ml of chloroform at 25° C. for 3 hours, followed by filtration with a 200-mesh stainless wire net, and an insoluble matter on the wire net was dried in vacuum. Next, the insoluble matter was precisely weighed and the gel fraction was calculated in percentage by the following formula.

Gel fraction (%)={weight of insoluble matter (mg)/weight of film weighed (mg)}×100 Formula (3)

[Biodegradable Test]

A 5B-type dumbbell test piece defined in ISO 527-2 was made with a punching die, 50 mL of a phosphate buffer saline solution (pH 7.4) was placed in a 50-mL sample bottle, and the dumbbell test piece was put and completely immersed therein. The sample bottle was placed in an oven at 50° C. and was allowed to stand for 2 weeks. The sample was taken out of the phosphate buffer saline solution and was immersed in ion exchange water at 37° C. to wash the sample. Then, the sample was quickly subjected to a tensile test using a tensile tester equipped with a thermostatic chamber (Autograph AG-1kNIS, from Shimadzu Corporation) under an atmosphere of 37° C. at a distance between chucks of 20 mm and a testing speed of 10 mm/min to measure the elongation at fracture. Separately, as a test specimen before hydrolysis, a sample was immersed in ion exchange water at 37° C. for 2 hours, then was taken out, and was quickly subjected to a tensile test. Finally, a ratio of the elongation at fracture after hydrolysis to the elongation at fracture before hydrolysis ((elongation at fracture after hydrolysis/elongation at fracture before hydrolysis)×100(%)) was determined.

The following Table 1 shows the polymer solution compositions, the evaluation results of the above evaluation, and Tg's (° C.) of the crosslinked polymers in Examples and Comparative Examples. The gel fractions in Examples were each 50% or more. For example, the gel fraction was 90% in Example 1, 52% in Example 9, and 66% in Example 11. Furthermore, each of the Examples exhibits biodegradability. For example, in Example 2, the elongation at fracture before hydrolysis test was 204%, the elongation at fracture after hydrolysis test was 122%, and the ratio of the elongation at fracture after hydrolysis test to the elongation at fracture before hydrolysis test was 60%. In Comparative Example 2, the elongation at fracture before hydrolysis test was 330%, the elongation at fracture after hydrolysis test was 295%, and the ratio of the elongation at fracture after hydrolysis test to the elongation at fracture before hydrolysis test was 89%.

TABLE 1

| | | Example | | | | | | | | | |
|---|---|---|---|---|---|---|---|---|---|---|---|
| | | 1 | 2 | 3 | 4 | 5 | 6 | 7 | 8 | 9 | 10 |
| Copolymer | Type | LCL7525 | LCL7525 | LCL7525 | LCL7525 | LCL7525 | LCL7525 | LCL7525 | LCL7525 | LCL7525 | LCL7525 |
| | SP value | 22.6 | 22.6 | 22.6 | 22.6 | 22.6 | 22.6 | 22.6 | 22.6 | 22.6 | 22.6 |
| | Molecular | 570,000 | 570,000 | 570,000 | 570,000 | 570,000 | 570,000 | 570,000 | 570,000 | 570,000 | 570,000 |
| | weight | | | | | | | | | | |
| | before | | | | | | | | | | |
| | crosslinking | | | | | | | | | | |
| | (Mw) | | | | | | | | | | |
| | Weight | 1 g | 1 g | 1 g | 1 g | 1 g | 1 g | 1 g | 1 g | 1 g | 1 g |
| Crosslinking | Type | PETA | PETA | PETA | PETA | PETA | DPEHA | DPEHA | EGDM | EGDM | EGDM |
| agent | SP value | 21.5 | 21.5 | 21.5 | 21.5 | 21.5 | 22.5 | 22.5 | 18.2 | 18.2 | 18.2 |
| | SP value | 1.1 | 1.1 | 1.1 | 1.1 | 1.1 | 0.1 | 0.1 | 4.4 | 4.4 | 4.4 |
| | difference*¹⁾ | | | | | | | | | | |
| | Weight | 0.1 g | 0.2 g | 0.3 g | 0.4 g | 0.5 g | 0.2 g | 0.4 g | 0.2 g | 0.1 g | 0.1 g |

| | | | | | | | | | | |
|---|---|---|---|---|---|---|---|---|---|---|
| Proportion by weight | 10% | 20% | 30% | 40% | 50% | 20% | 40% | 20% | 10% | 10% |
| of crosslinking agent | | | | | | | | | | |
| in copolymer (%) | | | | | | | | | | |
| Initiator | 0.01 g | 0.01 g | 0.01 g | 0.01 g | 0.01 g | 0.01 g | 0.01 g | 0.01 g | 0.01 g | — |
| Young's modulus (MPa) | 563 | 992 | 1126 | 1295 | 1386 | 641 | 1417 | 735 | 706 | 592 |
| Recovery rate after | 74 | 72 | 78 | 77 | 81 | 72 | 78 | 75 | 77 | 77 |
| 10 seconds (%) | | | | | | | | | | |
| Recovery rate after | 95.5 | 90.8 | 100 | 91.7 | 90.2 | — | — | — | — | — |
| 20 minutes (%) | | | | | | | | | | |
| Strain resistance property | ∘ | ∘ | ∘ | ∘ | ∘ | ∘ | ∘ | ∘ | ∘ | ∘ |
| Martens hardness (N/mm²) | 89 | 134 | 139 | 147 | 184 | 93 | 168 | 105 | 103 | 62 |
| Tg (° C.) | — | 40 | — | — | — | — | — | — | — | — |

Example

| | | 11 | 12 | 13 | 14 | 15 | 16 | 17 |
|---|---|---|---|---|---|---|---|---|
| Copolymer | Type | LCL9010 | LCL9010 | LCL9010 | LCL6535 | LCL6535 | LCL6535 | LCL6040 |
| | SP value | 22.9 | 22.9 | 22.9 | 22.4 | 22.4 | 22.4 | 22.3 |
| | Molecular weight | 530,000 | 530,000 | 530,000 | 320,000 | 320,000 | 320,000 | 370,000 |
| | before | | | | | | | |
| | crosslinking (Mw) | | | | | | | |
| | Weight | 1 g | 1 g | 1 g | 1 g | 1 g | 1 g | 1 g |
| Crosslinking | Type | PETA | PETA | PETA | PETA | PETA | PETA | PETA |
| agent | SP value | 21.5 | 21.5 | 21.5 | 21.5 | 21.5 | 21.5 | 21.5 |
| | SP value | 1.4 | 1.4 | 1.4 | 0.9 | 0.9 | 0.9 | 0.6 |
| | difference*¹⁾ | | | | | | | |
| | Weight | 0.1 g | 0.3 g | 0.4 g | 0.2 g | 0.3 g | 0.5 g | 0.4 g |

| | | | | | | | |
|---|---|---|---|---|---|---|---|
| Proportion by weight | 10% | 30% | 40% | 20% | 30% | 50% | 40% |
| of crosslinking agent | | | | | | | |
| in copolymer (%) | | | | | | | |
| Initiator | 0.01 g | 0.01 g | 0.01 g | 0.01 g | 0.01 g | 0.01 g | 0.01 g |
| Young's modulus (MPa) | 2028 | 1781 | 1640 | 511 | 655 | 880 | 502 |
| Recovery rate after 10 seconds (%) | 70 | 78 | 80 | 78 | 82 | 84 | 77 |
| Recovery rate after 20 minutes (%) | — | — | — | — | — | — | — |
| Strain resistance property | ∘ | ∘ | ∘ | ∘ | ∘ | ∘ | ∘ |
| Martens hardness (N/mm²) | 172 | 159 | 160 | 69 | 82 | 121 | 52 |
| Tg (° C.) | — | — | — | — | — | — | — |

Example

| | | 18 | 19 | 20 | 21 | 22 | 23 |
|---|---|---|---|---|---|---|---|
| Copolymer | Type | LCL7525 | LCL7525 | LCL7525 | LCL7525 | DLCL9010 | LT706S |
| | SP value | 22.6 | 22.6 | 22.6 | 22.6 | 22.9 | 22.6 |
| | Molecular weight | 570,000 | 570,000 | 570,000 | 570,000 | 180,000 | 250,000 |
| | before | | | | | | |
| | crosslinking (Mw) | | | | | | |
| | Weight | 1 g | 1 g | 1 g | 1 g | 1 g | 1 g |
| Crosslinking | Type | TAIC | TAIC | TAIC | TAIC | PETA | PETA |
| agent | SP value | 29.2 | 29.2 | 29.2 | 29.2 | 21.5 | 21.5 |
| | SP value | 6.6 | 6.6 | 6.6 | 6.6 | 1.4 | 1.1 |
| | difference*¹⁾ | | | | | | |
| | Weight | 0.1 g | 0.3 g | 0.5 g | 0.1 g | 0.4 g | 0.3 g |

| | | | | | | |
|---|---|---|---|---|---|---|
| Proportion by weight | 10% | 30% | 50% | 10% | 40% | 30% |
| of crosslinking agent | | | | | | |
| in copolymer (%) | | | | | | |
| Initiator | 0.01 g | 0.01 g | 0.01 g | — | 0.01 g | 0.01 g |
| Young's modulus (MPa) | 615 | 689 | 506 | 501 | 938 | 1358 |
| Recovery rate after 10 seconds (%) | 70 | 72 | 76 | 76 | 70 | 70 |
| Recovery rate after 20 minutes (%) | — | — | — | — | — | — |
| Strain resistance property | ∘ | ∘ | ∘ | ∘ | ∘ | ∘ |
| Martens hardness (N/mm²) | 62 | 99 | 66 | 51 | 127 | 156 |
| Tg (° C.) | — | — | — | — | — | — |

Comparative Example

| | | 1 | 2 | 3 | 4 | 5 | 6 |
|---|---|---|---|---|---|---|---|
| Copolymer | Type | PLLA | LCL7525 | LCL7525 | LCL7525 | LCL7525 | LT706S |
| | SP value | 23.1 | 22.6 | 22.6 | 22.6 | 22.6 | 22.6 |
| | Molecular | 510,000 | 570,000 | 570,000 | 570,000 | 570,000 | 250,000 |
| | weight before | | | | | | |
| | crosslinking | | | | | | |
| | (Mw) | | | | | | |
| | Weight | 1 g | 1 g | 1 g | 1 g | 1 g | 1 g |
| Crosslinking | Type | — | — | PETA | PETA | EGDM | — |
| agent | SP value | — | — | 21.5 | 21.5 | 18.2 | — |
| | SP value | — | — | 1.1 | 1.1 | 4.4 | — |
| | difference*¹⁾ | | | | | | |
| | Weight | — | — | 0.6 g | 0.05 g | 0.05 g | — |

| | | | | | | |
|---|---|---|---|---|---|---|
| Proportion by weight of | — | — | 60% | 5% | 5% | — |
| crosslinking agent in | | | | | | |
| copolymer (%) | | | | | | |
| Initiator | — | — | 0.01 g | 0.01 g | 0.01 g | — |
| Young's modulus (MPa) | 2127 | 375 | 1442 | 290 | 182 | 133 |
| Recovery rate after 10 | 57 | 71 | 82 | 84 | 53 | 63 |
| seconds (%) | | | | | | |
| Recovery rate after 20 | 75.2 | 87.8 | 86.5 | 83.8 | — | — |
| minutes (%) | | | | | | |
| Strain resistance property | x | ∘ | x | ∘ | ∘ | ∘ |
| Martens hardness (N/mm²) | 172 | 18 | 149 | 45 | 42 | 10 |
| Tg (° C.) | 65 | 37 | — | — | — | — |

*¹⁾Difference between solubility parameter of crosslinking agent and weighted average of solubility parameters of constitutional unit (A) and constitutional unit (B)

As can be seen from the foregoing results, the crosslinked polymers of the Examples each had a high Young's modulus, good strain resistance property, and a high 10-second recovery rate.

As can be seen from comparison of Example 10 and Example 21, when the absolute value of the difference between the solubility parameter of the crosslinking agent and the weighted average of the solubility parameters of the constitutional unit (A) and the constitutional unit (B) is 5 (J/cm³)^{1/2} or less, the Young's modulus is increased. EGDM used in Example 10 has two functional groups and has a higher Young's modulus than TAIC having three functional groups used in Example 21. That is, when TAIC or EGDM is used as the crosslinking agent, TAIC has a smaller effect of increasing the Young's modulus in spite of a larger number of functional groups (number of crosslinkable reaction points) in the added crosslinking agent, which suggests that the difference in solubility parameters is important. Similarly, by comparison of the results of Example 9 and Example 18, an increased Young's modulus leads to a higher shape recovery rate. That is, when TAIC or EGDM is used as the crosslinking agent, TAIC has a smaller effect of increasing the Young's modulus and the shape recovery rate in spite of a larger number of functional groups (number of crosslinkable reaction points) in the added crosslinking agent, which suggests that the difference in solubility parameters is important. Thus, when the absolute value of the difference between the solubility parameter of the crosslinking agent and the weighted average of the solubility parameters of the constitutional unit (A) and the constitutional unit (B) is 5 (J/cm³)^{1/2} or less, the effects of the embodiments (rapid shape recovery (high recovery rate) and high radial force (high Young's modulus)) are further easily obtained.

In contrast, Comparative Example 1 in which polylactic acid is used shows a low recovery rate in spite of a high Young's modulus. In addition, Comparative Example 2 in which L-lactic acid/ε-caprolactone copolymer (75/25) that is not crosslinked was used shows a low Young's modulus. As described in paragraph [0049] of Japanese Patent Application Publication No. 2015-527920 (International Patent Application Publication No. 2014/018123), Comparative Example 2 with an increased amount of the rubber-like polymer incorporated showed increased elasticity of the polymer and a relatively high 10-second recovery rate, while showing lowered mechanical strength.

Note that the recovery rates after 20 minutes in Examples 6 to 23 are 70% or more.

### Example 24

A tube was formed from a material of Example 1 and was subjected to laser cutting to fabricate a self-expandable stent (thickness: 150 μm, strut width: 150 μm, outer diameter: 3.5 mm (D1), length: 18 mm). The fabricated self-expandable stent was contracted in diameter and was inserted in a PTFE tube having an inner diameter of 1.2 mm. The tube was immersed in ion exchange water adjusted to 37° C., and the inserted self-expandable stent was released from the tube and was allowed to stand in ion exchange water at 37° C. for 1 minute. Then, the stent was taken out of water and the outer diameter (D2) was measured again with a caliper to calculate the shape recovery rate ((D2/D1)×100(%)).

The stent according to this Example showed a high shape recovery rate of 97%. This demonstrates that the stent can be suitably used as a self-expandable stent.

### Example 25, Comparative Example 7

A tube was formed from a material of Example 2 or Comparative Example 2 and was subjected to laser cutting to fabricate a self-expandable stent (thickness: 100 μm, strut width: 150 μm, outer diameter: 3.2 mm, length: 11 mm).

The shape recovery rate was measured in the same manner as in Example 24. The shape recovery rates of Example 25 and Comparative Example 7 were 97%.

In addition, using the self-expandable stent, the radial force was measured in accordance with ASTM F3067-14.

[Measurement Conditions]

Measurement apparatus: Radial Force Testing System-Model RFJ from Blockwise Engineering LLC

Measurement temperature: 37° C., speed (rate of diameter): 0.05 mm/sec

Measurement diameter: ϕ3.2 mm to ϕ1.5 mm

Measurement procedure: a sample is set in the apparatus heated to 37° C. While the diameter is decreased from ϕ3.2 mm to ϕ1.5 mm at a speed of 0.05 mm/sec and then is increased from ϕ1.5 mm to 0.2 mm, the radial force was measured.

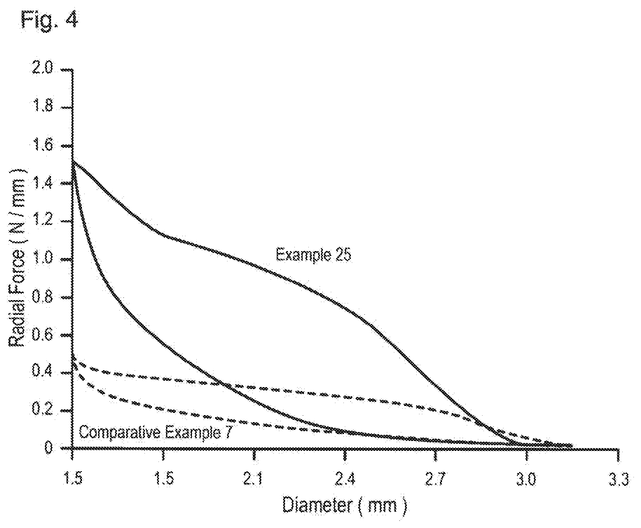

The results are disclosed in FIG. 4. As can be seen from FIG. 4, the stent of Example 25 showed significantly improved radial force compared to the stent of Comparative Example 7.

The detailed description above describes embodiments of a self-expandable stent and a method of producing a self-expandable stent representing examples of the inventive self-expandable stent and fabrication method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A self-expandable stent positionable in a living body and expandable from a contracted state while positioned in the living body, the stent comprising a plurality of wave-shaped struts positioned adjacent one another, with adjacent wave-shaped struts connected to one another by at least one connecting strut, the self-expandable stent being made of a material comprising:
constitutional unit (A), wherein constitutional unit (A) is a rigid biodegradable polymer derived from a first monomer selected from the group consisting of L-lactic acid, D-lactic acid and glycolic acid;
constitutional unit (B), wherein constitutional unit (B) is a rubber-like biodegradable polymer derived from a second monomer selected from the group consisting of ε-caprolactone, σ-butyrolactone, σ-valerolactone, 4-hydroxybytyrate, 3-hydroxybytyrate, 3-hydroxyvalerate, trimethylene carbonate, ethylene succinate, butylene succinate, and p-dioxanone; and
constitutional unit (C), wherein constitutional unit (C) is derived from a crosslinking agent.

2. The self-expandable stent according to claim 1, wherein the crosslinking agent is selected from the group consisting of pentaerythritol tetraacrylate, ditrimethylol propane tetraacrylate, dipentaerythritol hexaacrylate, dipentaerythritol monohydroxypentaacrylate, pentaerythritol tetramethacrylate, ditrimethylolpropane tetramethacrylate, dipentaerythritol hexamethacrylate, dipentaerythritol monohydroxypentamethacrylate, pentaerythritol tetraacrylate and dipentaerythritol hexaacrylate.

3. The self-expandable stent according to claim 1, wherein constitutional unit (C) is present in an amount that is equal to or greater than 10% by weight based on the total amount of constitutional units (A) and (B).

4. A self-expandable stent comprising a crosslinked polymer, wherein the crosslinked polymer comprises: constitutional unit (A), wherein constitutional unit (A) is a rigid biodegradable polymer derived from a first monomer; constitutional unit (B), wherein constitutional unit (B) is a rubber-like biodegradable polymer derived from a second monomer; and constitutional unit (C), wherein constitutional unit (C) is derived from a crosslinking agent, and wherein constitutional unit (C) is present in an amount that is equal to or greater than 10% by weight and less than 60% by weight based on the total amount of constitutional units (A) and (B).

5. The self-expandable stent according to claim 4, wherein the crosslinked polymer has a Young's modulus of 500 N/mm² or more and a recovery rate after 10 seconds of 70% or more.

6. The self-expandable stent according to claim 4, wherein the crosslinked polymer has a Martens hardness of 50 N/mm² or more in a loading-unloading test using a nanoindenter.

7. The self-expandable stent according to claim 4, wherein the crosslinked polymer is produced by polymerizing the crosslinking agent with a copolymer comprising constitutional unit (A) and constitutional unit (B), and
wherein an absolute value of a difference between a solubility parameter of the crosslinking agent and a weighted average of solubility parameters of the first monomer and the second monomer is 5 (J/cm³)^{1/2} or less.

8. The self-expandable stent according to claim 4, wherein the first monomer is lactic acid.

9. The self-expandable stent according to claim 4, wherein the second monomer is ε-caprolactone.

10. The self-expandable stent according to claim 4, wherein constitutional unit (B) is present in an amount 10 to 35% by mole based on the total amount of constitutional units (A) and (B).

11. The self-expandable stent according to claim 4, wherein the crosslinking agent is a multifunctional (meth)acrylate.

12. The self-expandable stent according to claim 11, wherein the multifunctional (meth)acrylate is a tetra- or higher functional (meth)acrylate.

13. A method of producing a self-expandable stent, comprising:
polymerizing a copolymer comprising constitutional unit (A), wherein constitutional unit (A) is a rigid biodegradable polymer derived from a first monomer and constitutional unit (B), wherein constitutional unit (B) is a rubber-like biodegradable polymer derived from a second monomer with a crosslinking agent, wherein the crosslinking agent is present in an amount that is equal to or greater than 10% by weight and less than 60% by weight based on the amount of the copolymer to produce a crosslinked polymer; and
fabricating the stent using the crosslinked polymer.

14. The method of producing a self-expandable stent according to claim 13, wherein the copolymer has an average molecular weight of 100,000 to 1,000,000.

15. The method of producing a self-expandable stent according to claim 13, wherein the copolymer and the crosslinking agent are polymerized under irradiation with an ultraviolet ray.

16. The method of producing a self-expandable stent according to claim 15, wherein the copolymer and the crosslinking agent are polymerized in the presence of a photoinitiator.

17. The method of producing a self-expandable stent according to claim 13, wherein the first monomer is selected from the group consisting of L-lactic acid, D-lactic acid and glycolic acid, and wherein the second monomer is selected from the group consisting of ε-caprolactone, σ-butyrolactone, σ-valerolactone, 4-hydroxybytyrate, 3-hydroxybytyrate, 3-hydroxyvalerate, trimethylene carbonate, ethylene succinate, butylene succinate, and p-dioxanone.

18. The method of producing a self-expandable stent according to claim 13, wherein the crosslinking agent is selected from the group consisting of pentaerythritol tetraacrylate, ditrimethylol propane tetraacrylate, dipentaerythritol hexaacrylate, dipentaerythritol monohydroxypentaacrylate, pentaerythritol tetramethacrylate, ditrimethylolpropane tetramethacrylate, dipentaerythritol hexamethacrylate, dipentaerythritol monohydroxypentamethacrylate, pentaerythritol tetraacrylate and dipentaerythritol hexaacrylate.

19. The method of producing a self-expandable stent according to claim 13, wherein the polymerizing is carried out in an organic solvent.

20. The method of producing a self-expandable stent according to claim 13, wherein the photoinitiator is selected from the group consisting of α-hydroxyalkylphenones, α-aminoalkylphenones, and acylphosphineoxide compounds.
